(19) United States
(12) Patent Application Publication
(10) Pub. No.: US 2025/0114751 A1
(43) Pub. Date: Apr. 10, 2025

(54) METHOD OF MEASURING CHEMICAL OXYGEN DEMAND (COD) IN SEAWATER

(71) Applicant: KING FAHD UNIVERSITY OF PETROLEUM AND MINERALS, Dhahran (SA)

(72) Inventors: Ahsan Mushir SHEMSI, Dhahran (SA); Safyan Akram KHAN, Dhahran (SA); Fahad Saleh AL-ISMAIL, Dhahran (SA)

(73) Assignee: KING FAHD UNIVERSITY OF PETROLEUM AND MINERALS, Dhahran (SA)

(21) Appl. No.: 18/480,842

(22) Filed: Oct. 4, 2023

(51) Int. Cl. B01D 61/46; G01N 1/44; G01N 33/18

(52) U.S. Cl. CPC B01D 61/463; G01N 1/44; G01N 33/1806; B01D 2311/04

(57) ABSTRACT

A method of measuring chemical oxygen demand (COD) in seawater including dialyzing the seawater including chloride ions and at least one organic compound with an electrodialysis unit to form a processed seawater having at least 25% less chloride ions than the seawater; mixing the processed seawater with an oxidizing agent to oxidize the at least one organic compound and heating to 100-200° C. for 1-5 hours to form a digested solution. The method further includes cooling the digested solution and adding an indicator to form a titration solution, and further performing a colorimetric titration on the titration solution with a reducing agent so as to reduce any excess of the oxidizing agent and reach an equivalence point based on the indicator. The COD in the seawater is calculated based on an amount of reducing agent used in the colorimetric titration.

### STATEMENT OF PRIOR DISCLOSURE BY INVENTOR

Aspects of the present disclosure are described in Shemsi, A. M. and Khan, S. A. and Alismail, F. “A Green Method for Determining the Chemical Oxygen Demand in Seawater by Removing Sodium Chloride Through Optimized Electrodialysis”; SSRN; Jun. 15, 2023, incorporated herein by reference in its entirety.

### STATEMENT OF ACKNOWLEDGEMENT

Support provided by King Fahd University of Petroleum and Minerals (KFUPM) is gratefully acknowledged.

### BACKGROUND

### Technical Field

The present disclosure is directed to a method of measuring chemical oxygen demand (COD) in seawater, particularly, to a method of measuring COD in the seawater using electrodialysis.

### Description of the Related Prior Art

The description of the related prior art provided herein is for the purpose of generally presenting the context of the disclosure. Work of the presently named inventors, to the extent described in this background section, as well as aspects of the description which may not otherwise qualify as prior art at the time of filing, are neither expressly nor impliedly admitted as prior art against the present invention.

Chemical oxygen demand (COD) measures the oxygen required to chemically oxidize all organic and inorganic matter in a water sample. Measuring the COD is essential for monitoring organic pollution in seawater and wastewater. It provides information on the amount of biodegradable and non-biodegradable organic matter present in water, affecting aquatic life's oxygen demand. High COD levels indicate a high concentration of organic matter, which can lead to eutrophication and other adverse effects on marine ecosystems.

One method for determining the COD of seawater is by spiking it with known amounts of glucose and potassium hydrogen phthalate (KHP), which are both oxidizable compounds. The sample is then incubated with a strong oxidizing agent, such as potassium dichromate, and the amount of oxygen consumed is measured. The COD can then be calculated by comparing the amount of oxygen consumed by the sample to that consumed by a known standard. This method, known as the Winkler method, is frequently used to test COD in wastewater and highly polluted surface waters because of its potent oxidizing activity, availability, repeatability, reproducibility, and ease of handling. However, chloride ions in the sample significantly affect the potassium dichromate method under acidic conditions and obscure its analysis, which can affect the measurement.

Another approach is to use a high-temperature digestion technique, such as the closed reflux method, which involves heating the seawater sample to a high temperature (approximately 150° C.) in the presence of a strong oxidizing agent, such as potassium dichromate. The heat and oxidizing agent break down the organic matter and release oxygen, which can then be measured. These methods do not apply to all types of seawater samples, and chloride ions can still react with the oxidizing agents used in COD analysis, leading to an overestimation of the COD value. Such interference is particularly significant in seawater and wastewater samples with high chloride concentrations collected from coastal and industrial processes, respectively.

Another method to overcome this interference is to use a modified version of the Winkler method. This method involves adding a reagent, such as manganese dioxide, to the seawater sample, which oxidizes organic matter and releases oxygen. Released oxygen is then collected and measured. The modified Winkler method is specifically designed to account for the presence of chloride ions, which can interfere with COD measurements. The primary disadvantage of the Winkler method is its low sensitivity; therefore, it may not detect low COD levels. In addition, it may not be suitable for specific sample types, such as those high in sulfur or sulfur compounds, because these can interfere with the accuracy of the test. Another disadvantage is that the Winkler method measures only the amount of oxygen consumed by inorganic matter, such as nitrate, sulfate, and chloride, and not by organic matter. Therefore, it does not detect certain organic pollutants, such as phenols and amino acids.

Furthermore, several other techniques have been developed to overcome chloride interference, including sample dilution to reduce the chloride content, determination of chemical oxygen demand of wastewaters without the use of mercury salts, and analyst pre-treatment with silver sulfate, and modification of the analytical method, such as adding silver sulfate as a catalyst or masking chloride with mercuric sulfate and magnesium sulfate. These chemicals can precipitate chloride ions and prevent their reaction with oxidizing agents. Other techniques, such as ion-exchange membranes, can also remove chloride ions from samples before COD analysis. Ion exchange can be applied to a small sample volume, making the analysis unreliable for seawater samples. These methods are presumably effective at reducing interference and improving the accuracy of COD measurements in seawater and wastewater samples.

Electrodialysis (ED) is a membrane separation technology that can remove chloride ions from seawater, making it an effective pre-treatment step for the accurate determination of COD in seawater samples. ED can selectively remove chloride ions from seawater, thus eliminating potential interference and improving the accuracy of the COD analysis. ED involves passing an electric current through a membrane that separates ions of different charges. The membrane allows ions of one charge to pass through while blocking ions of the opposite charge. When Cl⁻ is removed from the water, the membrane allows negatively charged ions such as chloride (Cl⁻) to pass through while blocking positively charged ions such as sodium (Na⁺).

ED typically involves two membrane types: cation-and anion-exchange membranes. Cation-exchange membranes are used to separate positively charged ions such as Na⁺, whereas anion-exchange membranes are used to separate negatively charged ions such as Cl⁻. The cation exchange membrane is placed on one side of the solution, and the anion exchange membrane is placed on the other side. An electric current is passed through the solution, attracting the Nations to the cathode and Cl⁻ ions to the anode. Chloride-depleted seawater can then be analyzed for COD using another method such as the potassium dichromate method.

Although various methods to measure COD in seawater samples have been developed, most of the conventional methods suffer from drawbacks, such as poor sensitivity and selectivity.

Hence, there is a need for a strategy to develop a simple and efficient strategy that may eliminate or overcome the limitations above. Therefore, it is one object of the present disclosure to provide a method of measuring COD in the seawater using electrodialysis.

### SUMMARY

In an exemplary embodiment, a method of measuring chemical oxygen demand (COD) in seawater is described. The method includes dialyzing the seawater including chloride ions and at least one organic compound with an electrodialysis unit to form a processed seawater having at least 25% less chloride ions than the seawater. The method further includes mixing the processed seawater with an oxidizing agent to oxidize the at least one organic compound and heating to 100-200° C. for 1-5 hours to form a digested solution. The method further includes cooling the digested solution and adding an indicator to form a titration solution. The method further includes performing a colorimetric titration on the titration solution with a reducing agent so as to reduce any excess of the oxidizing agent and reach an equivalence point based on the indicator. The method further includes calculating the COD in the seawater based on an amount of reducing agent used in the colorimetric titration. The digested solution and titration solution do not include a chloride masking agent. The electrodialysis unit includes an anode compartment, a first cationic membrane, a first buffer compartment, a first anionic membrane, a dialyzer compartment, a second cationic membrane, a second buffer compartment, a second anionic membrane, and a cathode compartment. Adjacent compartments are configured to allow passing of ions between compartments through anionic or cationic membranes. The anode compartment and the cathode compartment are connected to a power supply. The power supply provides a direct current voltage of 5-30 volts (V). The dialyzing includes pumping the seawater through the dialyzer compartment. The dialyzing includes pumping a buffer solution through the first buffer compartment and the second buffer compartment. The dialyzing includes pumping an electrolytic solution through the anode compartment and the cathode compartment. The pumping the seawater, the buffer solution, and the electrolytic solution are at a same or different flow rate between 1-10 milliliters per minute (mL/min). The electrolytic solution includes sodium sulfate and sulfuric acid each having a concentration of 0.01 to 1 molar (M).

In some embodiments, the power supply provides a direct current voltage of about 10 V.

In some embodiments, the method includes pumping the seawater, the buffer solution, and the electrolytic solution occur simultaneously.

In some embodiments, the pumping the seawater, the buffer solution, and the electrolytic solution are each with a separate peristaltic pump at a flow rate of about 5 mL/min.

In some embodiments, prior to the dialyzing a pH of the seawater is adjusted to less than 2.

In some embodiments, the electrolytic solution includes sodium sulfate and sulfuric acid having a concentration of about 0.5 M.

In some embodiments, the buffer solution includes seawater having a pH of less than 2.

In some embodiments, the oxidizing agent is selected from potassium permanganate, ozone, potassium dichromate, and manganese dioxide.

In some embodiments, the oxidizing agent is potassium dichromate, the indicator is ferroin and the colorimetric titration is performed with a ferrous ammonium sulfate solution.

In some embodiments, the chloride masking agent is selected from MgSO4, HgSO4, Al2 (SO4)3, Fe2(SO4)3, Ag2SO4, AgNO3, Cr3+, and Al3+.

In some embodiments, the first and second cationic membranes are the same or different and have an ion exchange capacity of 0.5-1.5 dry milliequivalents per gram (mEq/g), and a wet thickness of 0.01-0.1 millimeters (mm).

In some embodiments, the first and second anionic membranes are the same or different and have an ion exchange capacity of 1.5-3.0 dry meq/g, and a wet thickness of 0.05-0.5 mm.

In some embodiments, the COD concentration in the seawater is 5-1,000 milligrams per liter (mg/L).

In some embodiments, the limit of detection of the COD is 5 mg/L.

In some embodiments, the seawater has a salinity of 30-40 parts per thousand (ppt).

In some embodiments, the processed seawater has an electrical conductivity of less than 3 milliSiemens per centimeter (mS/cm).

In some embodiments, the dialyzing is for 1-500 minutes.

In some embodiments, the at least one organic compound is selected from petroleum, a dye compound, a pharmaceutical compound, a plasticizer, a humic compound, a phenolic compound, a surfactant, and a pesticide.

In some embodiments, the at least one organic compound is glucose.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic diagram showing the construction of an electrodialysis unit, according to certain embodiments of the present disclosure;

FIG. 1B is a flow chart of a method of measuring chemical oxygen demand (COD) in seawater, according to certain embodiments of the present disclosure;

FIG. 1C is a schematic diagram showing the construction of an electrodialysis unit, according to another embodiment of the present disclosure;

FIG. 2 depicts laboratory setup of the electrodialysis unit of FIG. 1C, according to certain embodiments of the present disclosure;

FIG. 3 is a graph depicting a change in sample conductivity at different applied voltages of 5, 10, 15, and 20 Volts (V), according to certain embodiments of the present disclosure;

FIG. 4 depicts a mechanism of electrodialysis and removal of sodium chloride from a seawater sample into a buffer through ion exchange membranes, according to certain embodiments of the present disclosure;

FIG. 5 is a graph depicting a change in the current and sample conductivity at an applied voltage of 10 V, with standard deviation (standard deviation calculated for N=4), according to certain embodiments of the present disclosure; and

FIG. 6 is a graph depicting a change in sample and buffer conductivities at an applied voltage of 10 V, according to certain embodiments of the present disclosure.

### DETAILED DESCRIPTION

When describing the present disclosure, the terms used are to be construed in accordance with the following definitions, unless a context dictates otherwise. Embodiments of the present invention will now be described more fully hereinafter with reference to the accompanying drawings wherever applicable, in which some, but not all embodiments of the disclosure are shown.

Further, as used herein, the use of singular includes plural and the words ‘a’, ‘an’ includes ‘one’ and means ‘at least one’ unless otherwise stated in this application.

Furthermore, the terms “approximately”, “approximate”, “about,” and similar terms generally refer to ranges that include the identified value within a margin of 20%, 10%, or preferably 5%, and any values therebetween.

The use of the terms “include,” “includes”, “including,” “have,” “has,” or “having” should be generally understood as open-ended and non-limiting unless specifically stated otherwise.

Where a numerical limit or range is stated herein, the endpoints are included. Also, all values and subranges within a numerical limit or range are specifically included as if explicitly written out.

As used herein, the term ‘COD’ refers to the chemical oxygen demand (COD) used as an indicative measure of the amount of oxygen that can be consumed by reactions in a measured solution.

According to an aspect of the present disclosure, a method of measuring chemical oxygen demand (COD) based on electrodialysis techniques is developed to remove the quantitative interference of chloride ions from seawater samples. The method does not use highly toxic masking agents such as mercuric sulfate to mask the interference of chloride ions in the COD determination. The results indicate that the method of the present disclosure can determine COD in seawater samples at low concentrations with a high degree of accuracy.

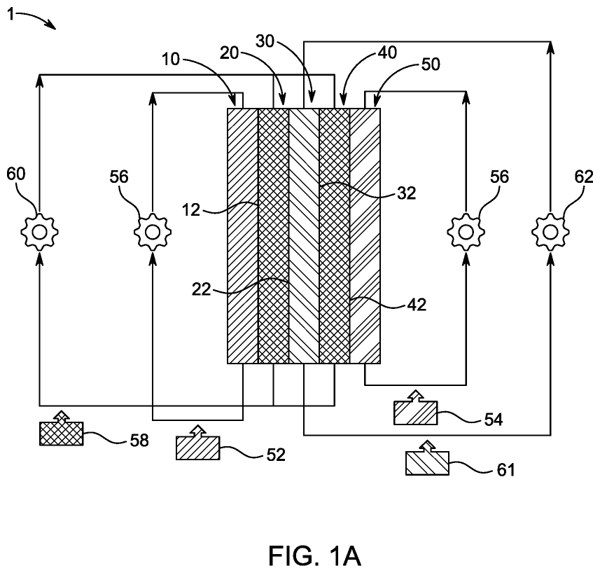

FIG. 1A illustrates a schematic diagram showing the construction of an electrodialysis unit 1 according to the present invention. The electrodialysis cell 1 includes an inlet configured to receive a feed. The feed includes seawater or saline solution. The electrodialysis unit 1 includes an anode compartment 10, a first cationic membrane 12, a first buffer compartment 20, a first anionic membrane 22, a dialyzer compartment 30, a second cationic membrane 32, a second buffer compartment 40, a second anionic membrane 42, a cathode compartment 50. Adjacent compartments are configured to allow the passing of ions between compartments through an anionic membrane (22 and 42) or the cationic membrane (12, and 32). The first cationic membrane 12 and the second cationic membrane 32 are collectively referred to as the cationic membrane, and the first anionic membrane 22 and the second anionic membrane 42 are collectively referred to as the anionic membrane.

In some embodiments, the first cationic membrane 12 and the second cationic membrane 32 are the same or different and have an ion exchange capacity of about 0.5 dry milliequivalents per gram (meq/g), 0.6 dry meq/g, 0.7 dry meq/g, 0.8 dry meq/g, 0.9 dry meq/g, 1.0 dry meq/g, 1.2 dry meq/g, 1.3 dry meq/g, 1.4 dry meq/g, and a wet thickness of about 0.01 millimeters (mm), about 0.02 mm, about 0.03 mm, about 0.04 mm, about 0.05 mm, about 0.06 mm, about 0.07 mm, about 0.08 mm, and about 0.09 mm. In some embodiments, the first cationic membrane 12 and the second cationic membrane 32 are the same or different and have an ion exchange capacity of about 0.5-1.5 dry meq/g, and a wet thickness of about 0.01 mm-0.1 mm.

In some embodiments, the first anionic membrane 22 and the second anionic membrane 42 are the same or different and have an ion exchange capacity of about 1.5 dry meq/g, about 1.6 dry meq/g, about 1.7 dry meq/g, about 1.8 dry meq/g, about 1.9 dry meq/g, about 2.0 dry meq/g, about 2.1 dry meq/g, about 2.2 dry meq/g, about 2.3 dry meq/g, about 2.4 dry meq/g, about 2.5 dry meq/g, about 2.6 dry meq/g, about 2.7 dry meq/g, about 2.8 dry meq/g, about 2.9 dry meq/g, and a wet thickness of about 0.05 mm, about 0.06 mm, about 0.07 mm, about 0.08 mm, about 0.09 mm, about 0.1 mm, about 0.2 mm, about 0.3 mm, about 0.4 mm. In some embodiments, the first and second anionic membranes 22, 42 are the same or different and have an ion exchange capacity of 1.5-3.0 meq/g, and a wet thickness of 0.05-0.5 mm. As used herein, the term ‘ion exchange capacity’ refers to the measure of a material's capability to displace ions formerly incorporated within its structure.

The alternating anionic and cationic membranes preferably have an accordion shape. For example, each membrane may have protrusions, such as folds, that extend into the cavity defined by a particular anionic/cationic membrane pair. The protrusion may extend to a depth of as much as 40% of the entire width of the cavity, preferably from 5-35% or 10-25% of the entire width of the cavity measured from the midpoint of opposing protrusions or folds of each, e.g., the membrane midpoint represents the position of the membrane if protrusion free. Protrusions or folds in the membranes serve to increase the surface area of the membrane and create an environment for turbulent flow within the cavity.

In some embodiments, multiple such electrodialysis units, preferably 2-10 units, may be connected in series to constitute the electrodialysis apparatus. A first container, such as but not limited to a beaker, 52 and a second container 54 containing an electrolytic solution is reticulated with first peristaltic pumps 56 (running at a specific speed) using tubing with the anode compartment 10 and the cathode compartment 50. A plurality of tubes circulate the electrolyte into the anode compartment 10 and the cathode compartment 50. A buffer solution in a third container 58 is connected to the first buffer compartment 20 and the second buffer compartment 40 of the electrodialysis unit 1 through a second peristaltic pump 60 using a tubing. A sample is placed in a container and coupled with the dialyzer compartment 30 through a third peristaltic pump 62 using tubing. A plurality of sensors are placed in the vials. The sensor may include, but are not limited to, pH, conductivity meter conductivity sensor. The first peristaltic pump 56, the second peristaltic pump 60, and the third peristaltic pump 62 may be operated at various flow rates.

The anode compartment 10 and the cathode compartment 50 of the electrodialysis unit 1 are connected to a power supply. The power supply provides a direct current voltage of greater than 0V, about 1 V, about 2 V, about 3V, about 4V, about 5 V, about 6 V, about 7 V, about 8 V, about 9 V, about 10 V, about 11 V, about 12 V, about 13 V, about 14 V, about 15 V, about 16 V, about 17 V, about 18 V, about 19 V, about 20 V, about 21 V, about 22 V, about 23 V, about 24 V, about 25 V, about 26 V, about 27 V, about 28 V, about 29 V. The power supply provides a direct current voltage upto 30 V. The power supply provides a direct current voltage of 5-30 V, more preferably 10 V.

FIG. 1B illustrates a flow chart of a method 70 of measuring chemical oxygen demand (COD) in seawater. The order in which the method 70 is described is not intended to be construed as a limitation, and any number of the described method steps can be combined in any order to implement the method 70. Additionally, individual steps may be removed or skipped from the method 70 without departing from the spirit and scope of the present disclosure.

At step 72, the method 70 includes dialyzing the seawater including chloride ions and at least one organic compound with the electrodialysis unit 1 to form a processed seawater. In some embodiments, the seawater has a salinity of 30 parts per thousand (ppt), 31 ppt, 32 ppt, 33 ppt, 34 ppt, 35 ppt, 36 ppt, 37 ppt, 38 ppt, 39 ppt, or 40 ppt. In some embodiments, the seawater has a salinity of 1-100 ppt, preferably 10 ppt, 20 ppt, 30 ppt, 40 ppt, 50 ppt, 60 ppt, 70 ppt, 80 ppt, or 90 ppt.

The seawater is dialyzed for about 1 minute, 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, 150 minutes, 160 minutes, 170 minutes, 180 minutes, 190 minutes, 200 minutes, 210 minutes, 220 minutes, 230 minutes, 240 minutes, 250 minutes, 260 minutes, 270 minutes, 280 minutes, 290 minutes, 300 minutes, 310 minutes, 320 minutes, 330 minutes, 340 minutes, 350 minutes, 360 minutes, 370 minutes, 380 minutes, 390 minutes, 400 minutes, 410 minutes, 420 minutes, 430 minutes, 440 minutes, 450 minutes, 460 minutes, 470 minutes, 480 minutes, and 490 minutes. The dialyzing is up to 500 minutes. In some embodiments, the dialyzing is for 1-500 minutes.

The dialyzing process is carried out by pumping the seawater through the dialyzer compartment 30. In some embodiments, prior to the dialyzing a pH of the seawater is adjusted to less than 2, preferably 1.9, 1.8, 1.7, 1.6, 1.5, 1.4, 1.3, 1.2, 1.1, 1.0, 0.9, 0.8, 0.7, 0.6, or 0.5. Any suitable acid may be used to adjust the pH such as but not limited to sulfuric acid, hydrochloric acid, or nitric acid.

In some embodiments, prior to the dialyzing 1-10 wt. % of a zeolite is added to the seawater to further absorb any Cl⁻ ions. Then prior to the dialyzing the seawater is filtered through a sieve to remove the zeolite. In some embodiments, prior to the dialyzing 1-10 wt. % of an ion-exchange polymer is added to the seawater. In some embodiments, the ion-exchange polymer is made of polystyrene sulfonate.

During the dialysis process, a buffer solution is pumped through the first buffer compartment 20 and the second buffer compartment 40; and an electrolytic solution is pumped through the anode compartment 10 and the cathode compartment 50. In some embodiments, the buffer solution includes seawater having a pH of less than 2, preferably 1.9, 1.8, 1.7, 1.6, 1.5, 1.4, 1.3, 1.2, 1.1, 1.0, 0.9, 0.8, 0.7, 0.6, or 0.5. Any suitable acid may be used to adjust the pH such as but not limited to sulfuric acid, hydrochloric acid, or nitric acid. In a preferred embodiment, the buffer solution is the same as the seawater solution that is utilized in the dialysis. In another embodiment, the buffer solution is sulfuric acid having a concentration of 0.01 molar (M), 0.02 M, 0.03 M, 0.04 M, 0.05 M, 0.06 M, 0.07 M, 0.08 M, and 0.09 M. The electrolytic solution includes sodium sulfate and sulfuric acid, each having a concentration of 0.01 M, 0.02 M, 0.03 M, 0.04 M, 0.05 M, 0.06 M, 0.07 M, 0.08 M, and 0.09 M. The electrolytic solution includes sodium sulfate and sulfuric acid, each having a concentration of 0.01-1 M. In some embodiments, the electrolytic solution includes sodium sulfate and sulfuric acid, having a concentration of about 0.5 M.

In some embodiments, the seawater, the buffer solution, and the electrolytic solution are pumped into the dialyzer compartment simultaneously. In some embodiments, the seawater, the buffer solution, and the electrolytic solution are pumped at a same or different flow rate between 1 milliliters per minute (mL/min), 2 mL/min, 3 mL/min, 4 mL/min, 5 mL/min, 6 mL/min, 7 mL/min, 8 mL/min, and 9 mL/min. The seawater, the buffer solution, and the electrolytic solution can be pumped at the same or different flow rate between 1-10 mL/min. In some embodiments, the seawater, the buffer solution, and the electrolytic solution are each pumped into the dialyzer compartment with the help of a separate peristaltic pump at a flow rate of about 5 mL/min.

In some embodiments, the at least one organic compound is selected from petroleum, a dye compound such as azo dyes such as acid orange 5, acid orange 7, acid orange 19, acid orange 20, acid red 13, acid red 88, alcian yellow, alizarine yellow R, allura red AC, amaranth (dye), amido black 10B, aniline yellow, arylide yellow, azo violet, basic red 18, biebrich scarlet, bismarck brown Y, black 7984, brilliant black BN, brown FK, brown HT, calconcarboxylic acid, chrysoine resorcinol, citrus red 2, congo red, disperse orange 1, eriochrome black T, fast yellow AB, hydroxynaphthol blue, janus green B, lithol rubine BK, methyl orange, mordant brown 33, naphthol AS, orange GGN, oil red O, pigment yellow 10, sunset yellow FCF, trypan blue, yellow 2G; azorubine, triarylmethane dyes such as 6-carboxyfluorescein, chlorophenol red, coomassie brilliant blue, cresol red, o-cresolphthalein, crystal violet; anthraquinone dyes such as anthrapyrimidine yellow (pigment yellow 108), anthraquinoid red (pigment red 177), and indanthrone blue (pigment blue 60); a pharmaceutical compound such as cetaminophen, metoprolol, caffeine, antipyrine, sulfamethoxazole, flumequine, ketorolac, atrazine, isoproturon, 2-hydroxybiphenyl, diclofenac, amitriptyline, and loperamide; a plasticizer such as glycerol (glycerin), propylene glycol, polyethylene glycols (PEG), Phthalate esters (diethyl, dibutyl), dibutyl sebacate, citrate esters (triethyl, acetyl triethyl, acetyl tributyl), triacetin, castor oil, acetylated monoglycerides, fractionated coconut oil; a humic compound, a phenolic compound such as vanillin, ferulic acid and p-coumaric acid; a surfactant such as cationic surfactants have cationic functional groups at their head, such as primary and secondary amines. The cationic surfactants include octenidine dihydrochloride; cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB). A cationic surfactant may be replaced by a nonionic surfactant, an anionic surfactant, a cationic surfactant, a viscoelastic surfactant, or a zwitterionic surfactant.

Anionic surfactants contain anionic functional groups at their head, such as sulfate, sulfonate, phosphate, and carboxylate. The anionic surfactant may be an alkyl sulfate, an alkyl ether sulfate, an alkyl ester sulfonate, an alpha olefin sulfonate, a linear alkyl benzene sulfonate, a branched alkyl benzene sulfonate, a linear dodecylbenzene sulfonate, a branched dodecylbenzene sulfonate, an alkyl benzene sulfonic acid, a dodecylbenzene sulfonic acid, a sulfosuccinate, a sulfated alcohol, a ethoxylated sulfated alcohol, an alcohol sulfonate, an ethoxylated and propoxylated alcohol sulfonate, an alcohol ether sulfate, an ethoxylated alcohol ether sulfate, a propoxylated alcohol sulfonate, a sulfated nonyl phenol, an ethoxylated and propoxylated sulfated nonyl phenol, a sulfated octyl phenol, an ethoxylated and propoxylated sulfated octyl phenol, a sulfated dodecyl phenol, and an ethoxylated and propoxylated sulfated dodecyl phenol. Other anionic surfactants include ammonium lauryl sulfate, sodium lauryl sulfate (sodium dodecyl sulfate, SLS, or SDS), and related alkyl-ether sulfates sodium laureth sulfate (sodium lauryl ether sulfate or SLES), sodium myreth sulfate, docusate (dioctyl sodium sulfosuccinate), perfluorooctanesulfonate (PFOS), perfluorobutanesulfonate, alkyl-aryl ether phosphates, and alkyl ether phosphates.

Zwitterionic (amphoteric) surfactants have both cationic and anionic groups attached to the same molecule. The zwitterionic surfactants include CHAPS (3-[(3-cholamidopropyl)dimethylammonio]-1-propanesulfonate), cocamidopropyl hydroxysultaine, ocamidopropyl betaine, phospholipids, and sphingomyelins.

Nonionic surfactants have a polar group that does not have a charge. These include long chain alcohols that exhibit surfactant properties, such as cetyl alcohol, stearyl alcohol, cetostearyl alcohol, oleyl alcohol, and other fatty alcohols. Other long chain alcohols with surfactant properties include polyethylene glycols of various molecular weights, polyethylene glycol alkyl ethers having the formula CH₃—(CH₂)₁₀₋₁₆—(O—C₂H₄)₁₋₂₅—OH, such as octaethylene glycol monododecyl ether and pentaethylene glycol monododecyl ether; polypropylene glycol alkyl ethers having the formula: CH₃—(CH₂)₁₀₋₁₆—(O—C₃H₆)₁₋₂₅—OH; glucoside alkyl ethers having the formula CH₃—(CH₂)₁₀₋₁₆—(O-glucoside)₁₋₃—OH, such as decyl glucoside, lauryl glucoside, octyl glucoside; polyethylene glycol octylphenyl ethers having the formula C₈H₁₇—(C₆H₄)—(O—C₂H₄)₁₋₂₅—OH, such as Triton X-100; polyethylene glycol alkylphenyl ethers having the formula C₉H₁₉—(C₆H₄)—(O—C₂H₄)₁₋₂₅—OH, such as nonoxynol-9; glycerol alkyl esters such as glyceryl laurate; polyoxyethylene glycol sorbitan alkyl esters such as polysorbate, sorbitan alkyl esters, cocamide MEA, cocamide DEA, dodecyldimethylamine oxide, block copolymers of polyethylene glycol and polypropylene glycol, such as poloxamers, and polyethoxylated tallow amine (POEA); and a pesticide such as malathion, acephate and disulfoton. In some embodiments, at least one organic compound is glucose.

In some embodiments, the processed seawater has at least 25% less chloride ions than the seawater before the dialysis, preferably 30%, 40%, 50%, 60%, 70%, 80%, 90%, 95% or 100% less chloride ions. In some embodiments, the processed seawater has an electrical conductivity of less than 3 millisiemens per centimeter (mS/cm), preferably 2.5, 2.4, 2.3, 2.2, 2.1, 2.0, 1.9, 1.8, 1.7, 1.6, 1.5, 1.4, 1.3, 1.2, 1.1, or 1.0 mS/cm. As used herein, the term ‘electrical conductivity’ refers to the measure of the capability of a material to pass the flow of electric current. Electrical conductivity differs from one material to another depending on the ability to let the electricity flow through them.

At step 74, the method 70 includes mixing the processed seawater with an oxidizing agent to oxidize the at least one organic compound and heating to about 100° C., 110° C., 120° C., 130° C., 130° C., 140° C., 150° C., 160° C., 170° C., 180° C., and 190° C. for about 1 hour, 2 hours, 3 hours and 4 hours to form a digested solution. In some embodiments, the method includes mixing the processed seawater with an oxidizing agent to oxidize the at least one organic compound and heating to about 100-200° C. for about 1-5 hours to form a digested solution. In some embodiments, the oxidizing agent is selected from potassium permanganate, ozone, potassium dichromate, and manganese dioxide. In some embodiments, the oxidizing agent includes hydrogen peroxide, peroxide, chromate, potassium permanganate, nitic acid, and nitrous oxide. In some embodiments, the oxidizing agent is potassium dichromate.

In a preferable embodiment of the invention, the oxidizing agent is added or mixed with the dialyzed sample in the form of a solid. For example, the oxidizing agent may be contacted with the dialyzed sample in the form of a compressed tablet. In a preferred embodiment, the oxidizing agent is mixed with the dialyzed sample in the form of a tablet that includes one or more inert soluble or insoluble diluents. The diluent is preferably an inorganic chloride free salt such as magnesium sulfate. The oxidizing agent is present in the tablet in combination with the diluent in an amount of less than 50 wt. %, preferably from 5-40 wt. %, or 10-25 wt. % of oxidizing agent based on the total weight of the dilutized composition. The diluent is preferably not a soluble organic material and preferably contains no organic or carbon-containing component. However, polymeric materials that are at least partially functionalized with one or more ionic groups are suitable. For example, a polymer such as a cellulose or substituted cellulose such as ethyl cellulose and/or methyl cellulose that is at least partially functionalized with one or more carboxylate groups is particularly effective for slow release of the oxidizing agent from its tabletized form without adding any oxidizable carbon load to the dialyzed sample. As the tablet disintegrates, the insoluble and/or carboxylate-functionalized cellulose-based polymer permits slow release of the oxidizing agent into the dialyzed sample. The slow release of the oxidizing agent achieved by mixing the oxidizing agent in the form of a dilutized solid composition with the dialyzed sample aids in avoiding over oxidation of carbon/organic materials present in the dialyzed sample solution. Mixing the oxidizing agent in its pure form directly with the dialyzed sample may lead to inefficient oxidation, inefficient allocation of the oxidizing agent and/or the formation of oxidation products at different stages of oxidation. The use of a dilutized solid form of the oxidizing agent avoids this problem.

The oxidizing agent when in tablet form preferably has a shape and structure that permits steady release of the oxidizing agent. In this regard a coin-shaped tablet having a ratio of diameter to height of 10:1, preferably 15:1, 20:1 or 25:1, is preferred. A large circular tablet that is thin and wide promotes even dissolution and distribution of the oxidizing agent over a wide area in a sample bottle or sample vessel that contains the dialyzed sample.

At step 76, the method 70 includes cooling the digested solution and adding an indicator to form a titration solution. In some embodiments, the indicator may include, but are not limited to, starch, phenolphthalein, ferrous ammonium sulfate, and ferroin. In some embodiments, the indicator is ferroin. The digested solution and the titration solution do not include a chloride masking agent. In some embodiments, the chloride masking agent is selected from MgSO₄, HgSO₄, Al₂(SO₄)₃, Fe₂(SO₄)₃, Ag₂SO₄, AgNO₃, Cr³⁺, and Al³⁺.

At step 78, the method 70 includes performing a colorimetric titration on the titration solution with a reducing agent so as to reduce any excess of the oxidizing agent and reach an equivalence point based on the indicator. As used herein, the term ‘colorimetric titration’ refers to the method of titration in which a titrating agent is used to determine the concentration of a chemical element or chemical compound in a solution with the aid of a color reagent or indicator. As used herein, the term ‘reducing agent’ refers to the reactants of an oxidation-reduction reaction which reduces the other reactant by giving out electrons to the reactant. In some embodiments, the colorimetric titration is performed with a ferrous ammonium sulfate solution.

At step 80, the method 70 calculating the COD in the seawater based on the amount of reducing agent used in the colorimetric titration. In some embodiments, the COD concentration in the seawater is 5-1,000 milligrams per liter (mg/L), preferably 50-900, 100-800, 200-700, 300-600, or 400-500 mg/L. In some embodiments, the limit of detection of the COD is 5 mg/L.

### EXAMPLES

The following examples demonstrate a method of measuring chemical oxygen demand (COD) in seawater described herein. The examples are provided solely for illustration and are not to be construed as limitations of the present disclosure, as many variations thereof are possible without departing from the spirit and scope of the present disclosure.

### Example 1: Materials

Analytical-grade potassium hydrogen phthalate (KC₈H₅O₄-KHP), glucose (C₆H₁₂O₆), potassium dichromate, ferrous ammonium sulfate, a ferroin indicator, silver sulfate, mercuric chloride, and sodium sulfate were obtained from Sigma-Aldrich. Ultrapure sulfuric acid was obtained from J. T. Bakers. pH and conductivity were measured using an Oakton PC 2700pH/conductivity meter. Chloride concentration was measured using a Dionex 3000 ion chromatograph coupled with a conductivity and UV detector. The total organic carbon (TOC) of the samples was measured using a Shimadzu TOC-V-CSN instrument. COD was measured following the standard methods for the Examination of Water and Wastewater 23^{rd} Edition (APHA 5220 C-COD): oxidation with dilute chromic acid and final titration with ammonium ferrous sulfate using ferroin as an indicator. The variable power supply used in the experiment was a Long Wei PS-3010DF, and the programmable peristaltic pumps were from Shenchen Lab V1. The water used in the experiments was obtained from an Elga Purelab Chorus, a deionized water purification system. The electrical resistance of deionized water was >18.2 mega Ohm-cm (0.055 μS-cm). The synthetic seawater used in the present disclosure was obtained from Merck (SSWS 500) and had a salinity of 37.979 ppt (%).

Different concentrations of the COD standards were prepared using potassium hydrogen phthalate and glucose. Potassium hydrogen phthalate and glucose were dried at 105° C. in an oven for 48 h and then placed in a desiccator for 24 hours until a constant weight was achieved. A stock solution of 1000 mg/L of COD was prepared by accurately weighing 0.8502 g of KHP and dissolving it in deionized water to a final volume of one liter. Similarly, a stock solution of 1000 mg/L of COD was prepared by accurately weighing 0.9375 g of glucose and dissolving it in one liter of water. Further standards of the required concentrations were prepared with appropriate dilutions of the stock solution in deionized water or seawater with pH<2.00, adjusted by adding sulfuric acid. These solutions were maintained at low temperatures (<4° C.), and fresh solutions were prepared weekly.

### Example 2: Design and Fabrication of Electrodialysis (ED) Cell

ED compartment cells were designed and fabricated at the King Fahd University of Petroleum and Minerals (KFUPM) workshop. The rectangular ED cell was fabricated from a 15 centimeters (cm) long, 5 cm wide, and 1 cm thick transparent polyacrylate (Perspex) block with a flow-through compartment that was 10 cm long, 1 cm wide, and 1 cm deep, tapered at the end with a volume of 10 cubic centimeters (cm³). Five such ED compartment cells were arranged in a cascade fashion, separated by alternating anion and cation membranes, and sandwiched between two stainless-steel 316 electrodes acting as the cathode and anode. These were connected with screws and nuts. The membrane types, fabrication, and properties are listed in Table 1. The central compartment acts as a sample dialyzer, the two outer compartments act as electrolytic solution compartments, and the two compartments sandwiched between the dialyzer and electrolytic compartments are the concentrators (buffers). A schematic of the ED assembly is presented in FIG. 1C.

TABLE 1
Specifications of electrodialysis membranes.

| | | Cationic | Anionic |
|---|---|---|---|
| S. No. | Properties | membrane | Membrane |
| 1 | Make | Solvay Fuel Cells | Huamtecho |
| | | Etc. | |
| | Model | Aquivion E 98-05 | TAEM 8040 |
| 2 | Wet Thickness (mm) | 0.05 | 0.15 |
| 3 | Ion exchange Capacity | 1 | 2.2 |
| | (dry meq/g) | | |
| 4 | Film surface resistance | — | 2.7 |
| | (Ohm cm², 0.5M | | |
| | NaCl, 25° C.) | | |
| 5 | pH | 1-14 | 1-12 |
| 6 | Water permeability | <0.1 | 0.1 |
| | (mL/hour · cm² MPa) | | |
| 7 | Selectivity | 100 | 98 |
| | permeability (%) | | |
| 8 | Thermal stability (° C.) | 80 | 50 |
| 9 | Bursting strength (MPa) | — | 0.25 |

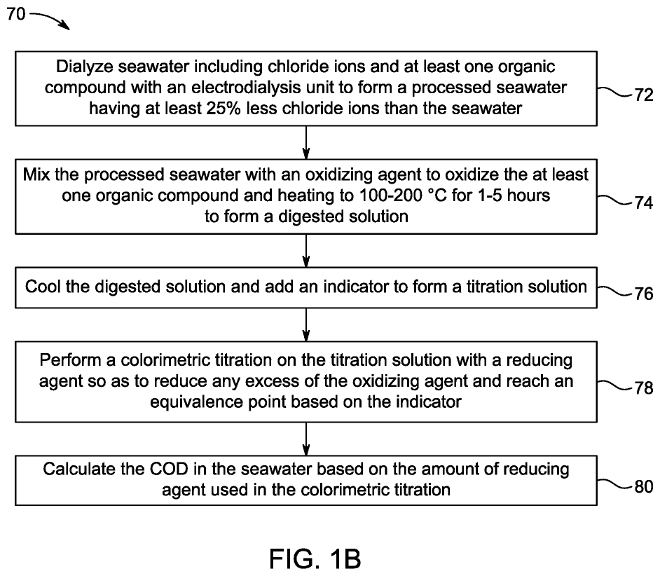

The ED compartment cells were arranged and hooked to four different solutions. Two beakers containing the electrolytic solution were reticulated with a peristaltic pump (running at a specific speed) using tubing with an internal diameter of 2 mm with anodic and cathodic chambers. The two tubes were used to circulate the electrolyte into two separate electrode chambers. The buffer solution in another beaker was connected to two compartments of the ED unit through a second peristaltic pump using the aforementioned tubing. The sample was placed in 50 mL polypropylene vials and coupled with the central sample dialyzer unit through the second peristaltic pump using peristaltic pump tubing. An Oakton (pH/conductivity meter) conductivity sensor was immersed in the vial. Both pumps were operated at various flow rates. The anode and cathode of the ED assembly were connected to a power supply (0-30 V, 0-6 ampere (A)). A schematic of the setup is shown in FIG. 2.

### Example 3: Sample Preparation

Synthetic seawater and seawater spiked with a known COD were processed in the ED unit. Fifty (50) mL of synthetic seawater whose pH was adjusted to <2.00 with the addition of sulfuric acid (concentrated) was placed in a 50 mL polypropylene vial supplied with the inlet and outlet tubes connected to the ED cell and were immersed in the liquid for the solution to be circulated between the cell and the ED cell using a peristaltic pump 1 (PP-1) at a selected (predefined) speed. A conductivity probe connected to an Oakton pH/conductivity detector was also immersed in this solution to continuously monitor its conductivity. Similarly, a beaker containing 100 mL of buffer solution was circulated through the two compartments around the sample dialyzer compartment using a PP-1. The two electrolytic compartments (anodic and cathodic) were fed with the electrolytic solution from two separate beakers (containing 100 mL of electrolyte each) using a separate peristaltic pump 2 (PP-2) and two separate tubing to keep the two solutions circulated separately. The power supply was switched on, and programmed in the constant-voltage mode set at the selected voltage. After ensuring that the solution in each compartment was circulated adequately throughout the setup, a power supply was connected to the two electrodes. The voltage, current, power, and conductivity of the samples were monitored at constant intervals until the electrical conductivity was reduced to 2 millisiemens (mS). The samples were preserved in glass vials for the total organic carbon (TOC) and COD analyses.

### Example 4: COD Analysis

The COD of the deionized water spiked with deionized water, synthetic seawater, spiked synthetic seawater, and processed seawater was determined following the Standard Methods for the Examination of Water and Wastewater 23^{rd} Edition (APHA method 5220 C) using dilute chromic acid and titration with ammonium ferrous sulfate. The Pyrex vial (16 mm×120 mm) and its Teflon-lined cap were thoroughly washed with 20% sulfuric acid and rinsed with deionized water, into which a 2.5 mL seawater sample was poured. To this sample, 0.9 g of solid HgSO₄, 3.5 mL of concentrated sulfuric acid, and 1.5 mL of 0.01667 M (0.1 N chromic acid) of digestion solution were added sequentially. The cap was tightly closed, and the tube was heated on a hot block at 150° C. for 2 h for chromic acid oxidation. After cooling the digested solution, the digested solution was transferred to a 50 mL beaker and washed. A ferroin indicator (0.1 mL) was added and titrated against 0.01 M of ferrous ammonium sulfate until the endpoint was reached. A seawater blank was prepared, digested, and titrated using the same method. For COD determination, all seawater samples were treated with HgSO₄ as the masking agent; however, deionized water and ED-processed samples were treated without the addition of the masking agent (HgSO₄). The following is the calculation for determining COD:

$$C O D in mg \frac{O 2}{L} = \frac{( A - B ) * M * 8000}{mL of samp1e}$$

### Example 5: TOC Analysis

Potassium hydrogen phthalate and glucose were used as the model compounds for seawater spiking and COD determination. Their concentrations in the prepared standards and processed samples were verified by determining the TOC. This was performed using a Shimadzu TOC analyzer following the Standard Methods for the Examination of Water and Wastewater, 23^{rd} Edition (APHA 5310 B-TOC). TOC standards of known concentrations in deionized and synthetic seawater were prepared for calibration. The TOC of the samples was determined for comparison with the COD and verification using the two methods.

### Example 6: Chloride Analysis

To monitor the chloride removal efficiency of the ED process, the concentration of chloride in the process samples was monitored by injecting the sample into an ion chromatograph equipped with guard and analytical columns, AG 19 and AS 19, respectively, following the US EPA method (EPA 300.1) and application note of the manufacturer. Samples were collected at the beginning and end of the experiment. To prevent the separating column from overloading, the seawater samples were diluted 10 times with deionized water, and 5 microliters (μL) were injected into the IC unit, whereas 25 μL of the processed samples were injected without dilution. The samples were eluted through the column with a self-generating KOH eluent at a concentration of 20 millimolar (mM), eluting the anions in the ion suppression mode at a flow rate of 1 mL/min for 20 min.

To determine the COD in seawater and remove chloride ions as interference, an ED process was applied, and the preferred process parameters were determined by varying the voltage, current density, electrolyte type, electrolyte concentration, buffer concentration (concentrate), and flow rate. The spiked concentration of COD in the synthetic seawater samples ranged from 5 to 50 mg/L. The direct current voltage was varied from 5 to 20 V, and the flow rate was varied from 1 to 10 mL/min. Sodium sulfate and sulfuric acid at various concentrations were used as electrolytes. The COD of the processed samples was determined using dilute 0.1 gram/liter (N) chromic acids without mercuric sulfate as a masking agent.

### Example 7: Effect of Voltage Variation

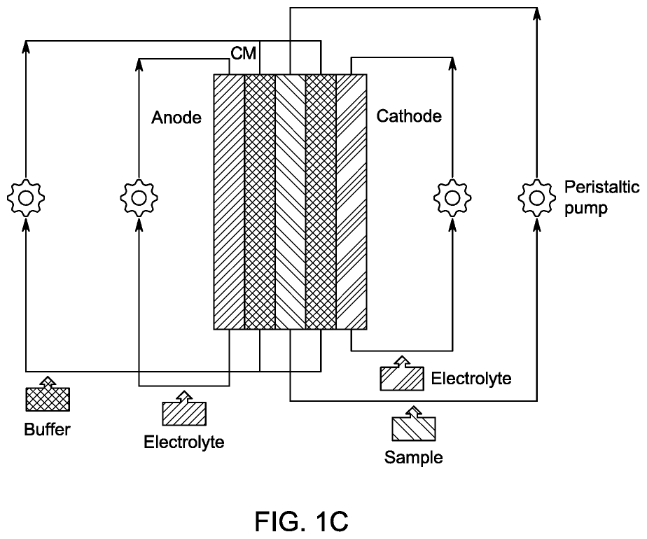

The electrical current passing through the solution was increased because of the increase in the voltage across the electrodes of the ED unit. This increase in the current positively influenced the ionic mobility of the ions in the solution. In the experiment, the voltage of the ED unit was varied between 5 V, 10 V, 15 V, and 20 V. FIG. 3 shows the influence of different applied voltages on the reduction in electrical conductivity of the seawater samples.

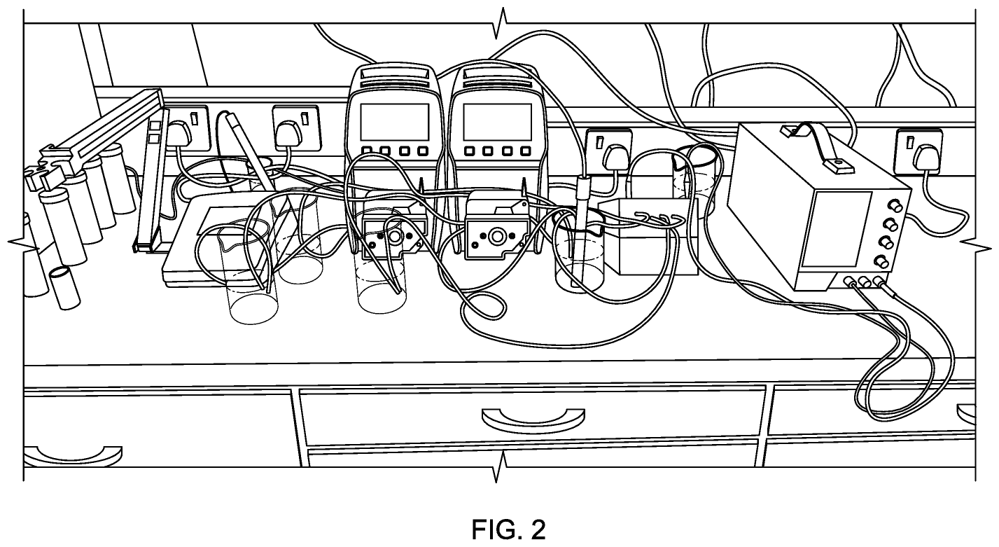

As the voltage increased, the current density in the solution also increased. This increase in current affected the removal of ions (cations and anions) from the solution. At lower voltages (5-10 V), the removal of ions takes more time compared to the higher voltages. At higher voltages, less time was required. However, unexpectedly at higher voltages (15 and 20 V), the experiment could not be continued because of the change in the pH of the test sample, which was initially adjusted to <2.0. It was noticed that the pH changed to 12 within 60 min and 90 min for the test samples at applied voltages of 20 V and 15 V, respectively. Due to the change in pH, a white salt precipitate was also observed. The appearance of a white precipitate due to the change in pH was verified by adding sodium hydroxide solution to a separate seawater sample to increase the pH to 12. The mechanisms of ED, removal of sodium chloride from seawater samples, and production of hydrogen and hydroxide ions are shown in FIG. 4. The following two reactions occur at the cathode and anode of the ED cell.

Reaction at Cathode (−): 2 H₂O+2 e⁻→H₂+2 OH⁻

Reaction at Anode (+): 2 H₂O→4 H⁺+4 e⁻+O₂

The change in pH at high voltages may be due to the leakage of OH⁻ ions across the membranes of the ED compartments produced at high concentrations at the cathode. The OH⁻ ions were sufficiently concentrated to not be removed from the cathodic chamber at the set flow rate (10 mL/minute). The ion removal efficiency at a low voltage (5 V) required more time than at 10 Volts. Therefore, further electrodialysis ED experiments on seawater samples were conducted at 10 V.

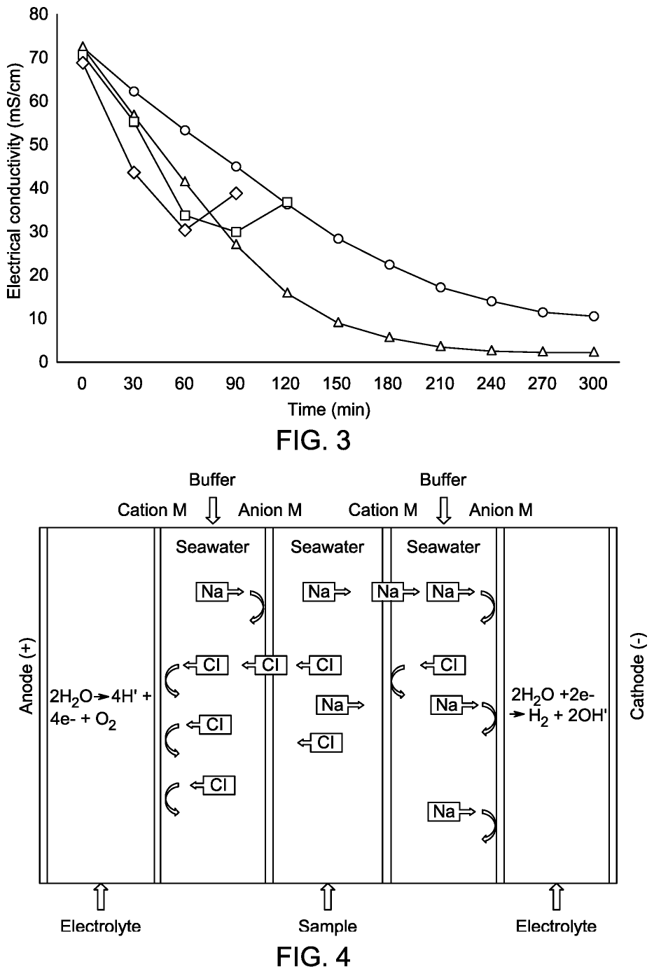

The changes in the electrical current and electrical conductivity of the synthetic seawater sample at a constant voltage of 10 V are shown in FIG. 5. It is noticeable that the electrical current and electrical conductivity of the samples decreased rapidly during the first 150 min. After 150 min, the electric current and electrical conductivity slowly reduced. This was due to the removal of cations and anions from the tested seawater sample, which increased the electrical resistance of the sample and decreased the electrical current and electrical conductivity. The electrical current in the entire electrodialysis unit containing the electrolyte, buffer, and test solution. However, only the electrical conductivities of the seawater samples were monitored. When the electrical conductivity of the tested seawater decreased to <2 millisiemens (mS) (approximately 270-300 min), the experiment was terminated. The samples were analyzed to determine their chloride, COD, and TOC contents. The chloride content and COD of the samples collected at various time intervals were also determined during the experiment.

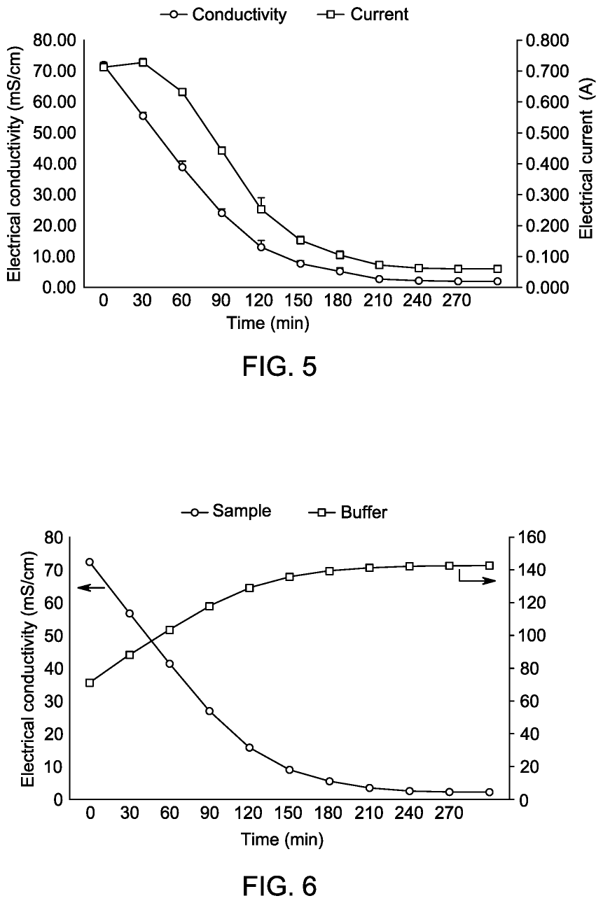

During the electrodialysis process, the anions and cations from seawater diffused across the cation and anion membranes into the adjacent buffer chambers, where a major ion exchange process occurred across the membranes. Therefore, the electrical conductivity of the buffer solution had to increase. The progress of cation and anion removal from seawater samples was monitored by measuring the electrical conductivity of the buffer solution. The increase in the electrical conductivity of the buffer and the decrease in the electrical conductivity of the sample are shown in FIG. 6. From FIG. 6, it is evident that the EC of the sample decreases from 72 millisiemens per centimeter (mS/cm) to <2 mS/cm. Correspondingly, the EC of the buffer doubled from 72 mS/cm to >140 mS/cm. Table 2 shows the initial and final pH values and the EC of the sample, buffer, and electrolyte for the experiment conducted at 10 volts.

TABLE 2
pH values and electrical conductivities of the initial and final
solution of sample, buffer, cathodic, and anodic chamber (electrolyte)
of the experiment conducted at 10 V for 300 min.

| | | | Conductivity | Conductivity final |
|---|---|---|---|---|
| Parameter | pH initial | pH final | Initial (mS/cm) | (mS/cm) |
| Sample | 1.29 | 1.20 | 71.55 | 2.195 |
| Buffer | 1.29 | 1.02 | 71.55 | 140.9 |
| Cathode | 1.21 | 1.02 | 211.01 | 76.47 |
| Anode | 1.21 | 1.02 | 211.01 | 143.4 |

The initial and final pH of the solutions did not change significantly (Table 2). However, the EC of the test samples decreased to 2.195 from 71.55 mS/cm. The EC of the buffer increased from 71.55 to 140.0 mS/cm. The increase in the EC of the buffer indicates that the ions that moved across the sample membranes did not diffuse further into the electrolytic solution (chamber). This can be understood from the schematic diagram (FIG. 4) for the ED unit. The membranes were configured alternatively in the ED unit. Therefore, the ions that moved into the buffer chamber could not diffuse further into the electrolytic chamber. This indicates that once the ions move to the buffer solution, the anions cannot further diffuse across the cation membrane, and the cations cannot move to the anion membrane in the corresponding electrolytic solution. Therefore, the EC of the buffer increases. This was observed in the experiment conducted at an applied potential of 10 V. The EC of the cathodic and anodic chamber electrolytes decreased from 211 to 76.47 mS and 211 to 143.4 mS, respectively. This may be due to the redox reaction occurring at the corresponding electrodes.

### Example 8: Effect of Electrolyte Concentration

Two electrolytes, namely, sulfuric acid and sodium sulfate of 0.1 and 0.5 M concentrations, were evaluated, and experimented trails were conducted to determine the preferred electrolyte during the ED process. At higher electrolyte concentrations, the current efficiency of the ED was higher than that at lower concentrations, which was directly proportional to the dialysis efficiency. This was verified by testing the chloride concentrations of the samples using ion chromatography, and their results are presented in Table 3. Increasing the concentration of the electrolyte resulted in an increase in current density and electrical conductivity, which has affected chloride removal efficiency. Sulfuric acid at higher concentrations was found to be more efficient compared to sodium sulfate in removing chloride from seawater. Owing to the corrosion of the stainless-steel electrode, the electrolyte solution of sulfuric acid changed from orange to red during the experiment. Similarly, in the case of sodium sulfate, a reddish-brown precipitate was produced because of the precipitation of sodium sulfate mixed with the corroding iron from the electrode. The sodium and sulfate present in seawater diffused across the membranes during ED, which caused a common ion effect on the sodium sulfate present in the electrolyte, resulting in the precipitation of sodium sulfate. Subsequently, sodium sulfate was deposited in the cell. The deposition of sodium sulfate affected the current density of the dialysis process and substantially reduced the chloride removal efficiency. However, in the case of sulfuric acid, the production of orange-red iron did not affect the chloride removal efficiency or current density. Therefore, only a higher concentration of sulfuric acid was used as the electrolyte in the experiment.

TABLE 3
Effect of different electrolytes and concentrations and
their effect on current density and chloride removal efficiency
at a constant voltage of 10 V after 120 minutes.

| | | | Current | |
|---|---|---|---|---|
| | | Conductivity | density | Cl removal |
| Electrolyte | mole/L | mS/cm | A | efficiency % |
| Sodium | 0.1 | 13.69 | 0.25 | 25.3 |
| sulfate | 0.5 | 27.46 | 0.47 | 45.54 |
| Sulfuric | 0.1 | 14.75 | 0.27 | 32.23 |
| acid | 0.5 | 28.1 | 0.49 | 51.65 |

### Example 9: Effect of Flow Rate

Three different flow rates (1, 5, and 10 mL/min) of the sample, buffer, and electrolyte in the ED experiment were tested. At a low flow rate of 1 mL/min, although the ion removal process was slightly faster, it took an extended amount of time to circulate the entire sample through the ED process. Moreover, the chlorine produced by the leakage of chloride ions in the electrolytic chamber produces chlorine gas, which may damage the membranes. Similarly, at a higher flow rate (10 mL/min), the ion removal was hindered by the short residence time of the test sample in the ED system. Therefore, a flow rate of 5 mL/min was used for the sample and buffer, and a flow rate of 10 mL/min was used for the electrolyte solution. This also helped remove the chlorine gas produced by the leakage of chloride ions in the electrolytic chamber.

### Example 10: Effect of Buffer Solution

Two buffers, diluted sulfuric acid, and seawater, were used for the experiment. Although they do not act as buffers, they act as a buffer solution between the electrolyte and the sample. The purpose of the buffer is to receive ions from the samples and to act as a pre-concentrator. Sulfuric acid with a concentration of 0.5 M and synthetic seawater at 50% dilution was used at an applied voltage of 10 V. There was a minimal difference in the ion removal efficiency when these two buffer types were used. Therefore, 100% synthetic or field-collected filtered and acidified seawater was used as a buffer to prevent the generation of spent sulfuric acid as waste. Moreover, it was assumed that using the same seawater as both the buffer and sample did not affect the concentration of COD in the seawater sample. Therefore, in further experiments, 100% seawater, whose pH was adjusted to <2.00 with sulfuric acid, was used as a buffer or concentrator.

### Example 11: Application of the Determined Parameters on the COD Analysis in Seawater

The results of seven replicates of synthetic seawater spiked with varying amounts of KHP in a solution whose pH was adjusted to <2.00 with sulfuric acid were subjected to ED at an applied voltage of 10 V, and the processes for 300 min are presented in Table 4. After processing, the samples were analyzed for COD with and without HgSO₄ as a masking agent. For verification, the COD, TOC, and chloride contents were also measured. The table shows the amount of phthalate used to prepare the appropriate COD concentration in the seawater samples. The theoretical COD concentration was compared with the experimental value determined in the same solution after processing using the ED process. The results for different COD ranges are presented as the standard error and percentage recovery of seven samples. Phthalate is ionic and carries a negative charge above pH 5.4 (pKa 5.4). However, at pH<2.00, it was neutral and did not move across the membrane during ED. The purpose of maintaining a pH<2 was based on the COD sample preservation method, in addition to running the ED process. Similarly, all humic acids (a major contributor to COD) present in the wastewater and seawater appeared neutral.

TABLE 4
Chemical oxygen demand and total organic carbon of KHP of a
synthetic seawater sample were determined with the ED method.

| | Concentration | COD | COD | Std. | TOC | TOC |
|---|---|---|---|---|---|---|
| | of Phthalate | (Calc) | (Exp) | Error | (Calc) | (Exp) |
| Sr. No. | mg/L | mg/L | mg/L | mg/L | mg/L | mg/L |
| 1 | 0 | 0 | 0.04 | 0.02 | 0 | 0 |
| 2 | 4.125 | 5 | 4.97 | 0.04 | 2 | 2.1 |
| 3 | 8.25 | 10 | 10 | 0.03 | 4 | 5.44 |
| 4 | 20.625 | 25 | 25 | 0.05 | 10 | 10.88 |
| 5 | 41.25 | 50 | 50.01 | 0.02 | 20 | 18.96 |

Std error calculated for N = 7

The results of seven replicates of synthetic seawater spiked with varying amounts of glucose, processed with ED to remove chloride, and analyzed as described above are presented in Table 5. The pKa of glucose is 12, and at pH<2.00, it is neutral and does not move across membranes during ED. The results indicate that the standard error was very low.

The purpose of spiking seawater with two organic compounds was to demonstrate the application of this technique to simple (linear and cyclic hydrocarbons) and complex (aromatic) organic compounds. The analysis of COD using alkaline KMnO₄ to oxidize glucose and KHP have shown its inability to oxidize these compounds (50% and 20%, respectively) (Liu, L. et al. (2005) ‘Chemical oxygen demand of seawater determined with a microwave heating method’, Journal of Ocean University of China, 4(2), pp. 152-156). Therefore, this method is not accepted globally, and there is a need to modify and improve the chromic acid method for its applicability to saline water at a lower concentration range of COD (<20 mg/L). The minimum detection limit obtained using ED for 180 min was 5.0 mg/L for glucose and phthalates.

TABLE 5
Chemical Oxygen Demand and Total Organic carbon of glucose of
a synthetic seawater sample determined using the ED method.

| | Glucose | COD | COD | Std. | TOC | TOC |
|---|---|---|---|---|---|---|
| | Concentration | (Calc) | (Exp) | Error | (Calc) | (Exp) |
| Sr. No. | mg/L | mg/L | mg/L | mg/L | mg/L | mg/L |
| 1 | 0 | 0 | 0.04 | 0.01 | 0 | 0 |
| 2 | 4.695 | 5 | 5.02 | 0.01 | 1.87 | 1.95 |
| 3 | 9.389 | 10 | 10 | 0.01 | 3.75 | 4.11 |
| 4 | 23.474 | 25 | 24.98 | 0.01 | 9.37 | 9.56 |
| 5 | 46.948 | 50 | 50.01 | 0.01 | 18.73 | 18.84 |

Std error calculated for N = 7

Samples collected from different locations in the Arabian Gulf were also subjected to ED, and further analysis of COD following the above-mentioned method is presented in Table 6.

Although the calibration of the COD was performed in the range of 5 to 50 mg/L, the COD of the collected sample was found to be lower than the calibration range. The samples were spiked with the minimum COD standard of 5 mg/L of the phthalate, and the percent recoveries of these samples were in the range of 96 to 102%, which is in good agreement. From the COD results of the processed and spiked samples, it is confirmed that the new method can be successfully applied for the determination of the COD of seawater samples at low concentrations without using HgSO₄.

TABLE 6
Chemical Oxygen Demand for seawater samples from
the Arabian Gulf spiked with known COD concentrations
of 5 mg/L and their recoveries.

| | | COD | Percent |
|---|---|---|---|
| | | (exp) | Recovery |
| Sr. No. | Location | mg/L | (%) |
| 1 | Dammam | 3.51 | — |
| 2 | Dammam SPK* | 8.45 | 98.8 |
| 3 | Halfmoon Beach | 1.22 | — |
| 4 | Halfmoon Beach | 6.3 | 101.6 |
| | SPK* | | |
| 5 | Tarut Bay | 2.56 | — |
| 6 | Tarut Bay SPK* | 7.4 | 96.8 |
| 7 | Al-Jubail | 3.3 | — |
| 8 | Al-Jubail SPK* | 8.22 | 98.4 |
| 9 | Safaniya | 1.22 | — |
| 10 | Safaniya SPK* | 6.28 | 101.2 |

Sample spiked with 5 mg/L COD (KHP)

The determination of COD in saline water (wastewater and seawater) is often influenced by the presence of chloride ions, which are masked using highly toxic HgSO₄. The use of HgSO₄ is not a safe process because an enormous amount of toxic waste is generated. A self-designed, locally fabricated electrodialysis unit containing five compartments was fabricated to successfully remove sodium chloride from a saline solution and determine the COD of wastewater containing high chloride content and seawater. The feasible process parameters in terms of the electrolyte types, electrolyte concentration, concentrator types, and electrolyte concentration, applied voltage, flow rate, and time were determined; and these parameters were applied to seawater samples with a known spiked COD concentration. The method of the present disclosure overcomes the challenges associated with chloride ions interference and is also effective in determining low levels of COD as low as 5 mg/L without using HgSO₄ as a masking agent.

Numerous modifications and variations of the present disclosure are possible in light of the above teachings. It is, therefore, to be understood that within the scope of the appended claims, the invention may be practiced otherwise than as specifically described herein.

1. A method of measuring chemical oxygen demand (COD) in seawater, comprising:
dialyzing the seawater comprising chloride ions and at least one organic compound with an electrodialysis unit to form a processed seawater having at least 25% less chloride ions than the seawater;
mixing the processed seawater with an oxidizing agent to oxidize the at least one organic compound and heating to 100-200° C. for 1-5 hours to form a digested solution;
cooling the digested solution and adding an indicator to form a titration solution;
performing a colorimetric titration on the titration solution with a reducing agent so as to reduce any excess of the oxidizing agent and reach an equivalence point based on the indicator; and
calculating the COD in the seawater based on an amount of reducing agent used in the colorimetric titration,
wherein the digested solution and titration solution do not comprise a chloride masking agent,
wherein the electrodialysis unit, comprises in order:
an anode compartment;
a first cationic membrane;
a first buffer compartment;
a first anionic membrane;
a dialyzer compartment;
a second cationic membrane;
a second buffer compartment;
a second anionic membrane; and
a cathode compartment;
wherein adjacent compartments are configured to allow passing of ions between compartments through anionic or cationic membranes,
wherein the anode compartment and the cathode compartment are connected to a power supply,
wherein the power supply provides a direct current voltage of 5-30 volts (V), and
wherein the dialyzing comprises:
pumping the seawater through the dialyzer compartment;
pumping a buffer solution through the first buffer compartment and the second buffer compartment; and
pumping an electrolytic solution through the anode compartment and the cathode compartment,
wherein the pumping the seawater, the buffer solution, and the electrolytic solution are at a same or different flow rate between 1-10 milliliters per minute (mL/min), and
wherein the electrolytic solution comprises sodium sulfate and sulfuric acid each having a concentration of 0.01 to 1 molar (M).

2. The method of claim 1, wherein the power supply provides a direct current voltage of about 10 V, and
wherein at least one of the first and second anionic and cationic membranes has an accordion shape with protrusions that extend to a depth up to 40% of an entire width of a cavity.

3. The method of claim 1, wherein the pumping the seawater, the buffer solution, and the electrolytic solution occur simultaneously.

4. The method of claim 1, wherein the pumping the seawater, the buffer solution, and the electrolytic solution are each with a separate peristaltic pump at a flow rate of about 5 mL/min.

5. The method of claim 1, wherein prior to the dialyzing a pH of the seawater is adjusted to less than 2.

6. The method of claim 1, wherein the electrolytic solution comprises sodium sulfate and sulfuric acid having a concentration of about 0.5 M.

7. The method of claim 1, wherein the buffer solution comprises seawater having a pH of less than 2.

8. The method of claim 1, wherein the oxidizing agent is selected from potassium permanganate, ozone, potassium dichromate, and manganese dioxide.

9. The method of claim 1, wherein the oxidizing agent is potassium dichromate, the indicator is ferroin and the colorimetric titration is performed with a ferrous ammonium sulfate solution.

10. The method of claim 1, wherein the chloride masking agent is selected from MgSO₄, HgSO₄, Al₂(SO₄)₃, Fe₂(SO₄)₃, Ag₂SO₄, AgNO₃, Cr³⁺, and Al³⁺.

11. The method of claim 1, wherein the first and second cationic membranes are the same or different and have an ion exchange capacity of 0.5-1.5 dry milliequivalents per gram (mEq/g), and a wet thickness of 0.01-0.1 millimeters (mm).

12. The method of claim 1, wherein the first and second anionic membranes are the same or different and have an ion exchange capacity of 1.5-3.0 dry meq/g, and a wet thickness of 0.05-0.5 mm.

13. The method of claim 1, wherein the COD concentration in the seawater is 5-1,000 milligrams per liter (mg/L).

14. The method of claim 1, wherein the limit of detection of the COD is 5 mg/L.

15. The method of claim 1, wherein the seawater has a salinity of 30-40 parts per thousand (ppt).

16. The method of claim 1, wherein the processed seawater has an electrical conductivity of less than 3 milliSiemens per centimeter (mS/cm).

17. The method of claim 1, wherein the dialyzing is for 1-500 minutes.

18. The method of claim 1, wherein the at least one organic compound is selected from petroleum, a dye compound, a pharmaceutical compound, a plasticizer, a humic compound, a phenolic compound, a surfactant, and a pesticide.

19. The method of claim 1, wherein the at least one organic compound is glucose.

20. The method of claim 1, wherein the oxidizing agent is mixed with the processed seawater in a form of a compressed tablet,
wherein the compressed tablet includes a magnesium sulfate diluent and 0.1-50 wt. % of the oxidizing agent based on a total weight of the compressed tablet, and
wherein the tablet has a coin shape having a ratio of diameter to height of 5:1 to 10:1.
